(19) United States
(12) Patent Application Publication
(10) Pub. No.: US 2001/0006694 A1
(43) Pub. Date: Jul. 5, 2001

(54) DRY FOODSTUFF WITH IMPROVED FLAVOUR CONTAINING DIPEPTIDE SWEETENER

(76) Inventors: JOHN FRY, HORSHAM (GB); ANNETTE HOEK, KERKRADE (NL); LEOPOLD F. W. VLEUGELS, BEEK (NL)

Correspondence Address: PILLSBURY MADISON & SUTRO, INTELLECTUAL PROPERTY GROUP, 1100 NEW YORK AVENUE N W, NINTH FLOOR EAST TOWER, WASHINGTON, DC, 200053918

(21) Appl. No.: 09/233,159

(22) Filed: Jan. 19, 1999

Related U.S. Application Data
(63) Continuation of application No. PCT/NL97/00415, filed on Jul. 14, 1997.

(30) Foreign Application Priority Data
Jul. 16, 1996 (NL) 1003604

(51) Int. Cl. A61K 9/68; A23L 1/236; A23L 1/00

(52) U.S. Cl. 426/548; 426/804; 530/801

(57) ABSTRACT

The invention relates to dry foodstuffs with <10% by wt moisture, containing dipeptide sweetener and optionally a component that is reactive with it, both in a sugar-free and in a sugar-containing form. The invention relates especially to products which also contain flavourings based on aldehydes. With the invention dry foodstuffs are produced with improved sweetness stability and storage stability and improved flavour when the dipeptide sweetener is a sweetening salt of an aspartic-acid-derived dipeptide sweetener and a derivative of a sweetening acid. The particle size of the sweetening salt is preferably smaller than 200 μm. The invention also relates to processes for production of such dry foodstuffs using the dipeptide sweetener substantially in solid, dry form.

The invention relates to dry foodstuffs containing dipeptide sweetener. The term “dry foodstuffs” as used in this application includes all possible dry foodstuffs, that is to say foodstuffs with a low moisture content, in particular <10% by wt, which are sweetened with dipeptide sweetener. The dry foodstuffs can be available both in a sugar-free and in a sugar-containing form. The invention relates more particularly to dry foodstuffs which are produced in substantially solid, dry form using the dipeptide sweetener. The term “dry foodstuffs” also includes chewing gums and all other forms of chewable gum-based products sweetened with dipeptide sweetener. The invention is intended to improve the sweetness stability and the flavour properties of such dry foodstuffs, especially insofar as the stability during storage (storage stability) and the flavour quality are concerned. In particular the invention also relates to dry foodstuff products sweetened with a dipeptide sweetener which contain flavourings based on aldehydes (i.e. the so-called aldehyde flavour products, or aldehyde flavourings), and also to products in which in addition to the dipeptide sweetener there are also components which can react with the dipeptide sweetener, such as vitamin C ((dehydro)ascorbic acid), uronic acids, strongly reducing sugars, for example D-glucose, D-fructose, D-mannose, D-galactose, D-xylose, L-arabinose and D-ribose, (less) reactive reducing sugars such as lactose, cellobiose, maltose and sucrose, and also products such as sodium bicarbonate, etc. The term “aroma substances” is sometimes used instead of “flavourings”.

Examples of dry foodstuffs as meant in the context of this application are powdered mixtures for instant lemonades or for other drinks based on coffee, tea, cocoa or milk, powdered mixtures for instant desserts based on water or milk, powdered mixtures for making ice cream, powdered mixtures for making dietetic meals, pharmaceutical powdered mixtures and compressed pharmaceutical tablets, hard confectionery such as fruit, menthol and mint pastilles or fruit, menthol and mint sweets and fruit, menthol and mint drops, chocolate, powdered confectionery, effervescent tablets, (effervescent) tablets for making soft drinks, sweetening agents in the form of sweetening tablets, low-calorie sugar cubes or powdered table sweeteners, chewing gums and chewing tablets.

Dry foodstuffs sweetened with dipeptide sweetener are known and described in various patent publications, for example in WO-92/07473. This describes dry foodstuffs, in this case chewing gums, in which longer and improved flavour is achieved by using coated synergistic mixtures of the two intense sweeteners aspartame and acesulfame-K; it also reports that there are less marked effects as regards stability problems which can arise with aspartame in the presence of aldehydes, ketones, moisture, etc. Maillard reactions, for example, can occur—which can lead to loss of sweet flavour and the development of unwanted off-flavours and/or after-flavours. This is already detrimental during the manufacture of the products, but also detrimental to the storage stability of the products in which the said components are present together, as the sweetening power decreases as a result. Under such circumstances, as a result of unwanted reactions, there may also be discoloration of the (solid) products in which a dipeptide sweetener is incorporated. The reactions in question take place mainly during the manufacture of the foodstuffs and during the storage thereof if there is increased or relatively high temperature, or increased or relatively high humidity. In addition, in dry foodstuffs containing dipeptide sweetener, regardless of the presence or absence of components that are reactive with a dipeptide sweetener, there often is a need for improved flavour and sweetness and more homogeneous distribution of such improved flavour and sweetness through the foodstuff.

Dry foodstuffs sweetened with dipeptide sweetener are commercially available, in various flavours, including aldehyde flavour, such as chewing gum products from the Wrigley range, e.g. Wrigley's Extra PlenTpak® with cinnamon flavour. Another example can be found in Sanatogen® vitamin C tablets; these contain inter alia sodium bicarbonate, ascorbic acid and aspartame. A variety of chocolates sweetened with dipeptide sweetener are also known. There are many other examples.

For the purposes of this application “dipeptide sweetener” means products with a sweetening power which is many dozens of times stronger than that of sugar, and which are made up of amino acids or derivatives of amino acids linked via a peptide bond. The best-known examples of dipeptide sweeteners are aspartame and alitame.

Aspartame (α-L-aspartyl-L-phenylalanine methyl ester), hereinafter also referred to as APM, is a dipeptide sweetener with a sweetening power which is approximately 200× that of sucrose. Aspartame is used as an intense sweetener in many applications because of its good flavour properties and low-calorie characteristics. Aspartame is also widely used in dry foodstuffs.

Alitame (L-α-aspartyl-N-(2,2,4,4-tetramethyl-3-thietanyl)-D-alanineamidehydrate), hereinafter also referred to as ALI, is a dipeptide sweetener with a sweetening power which is approximately 2000× that of sucrose.

It is also known that dipeptide sweeteners in dry foodstuffs are used in the form of blends, i.e. physical mixtures, with other sweeteners. See for example Patent WO-92/07473, already mentioned above, in which a (coated) blend of APM and acesulfame-K (the potassium salt of 6-methyl-1,2,3-oxathiazin-4(3H)-one-2,2-dioxide, hereinafter also referred to as Ace-K) is used.

As will be apparent from the foregoing, the use of dipeptide sweeteners in dry foodstuffs, in particular in dry foodstuffs which contain flavourings based on aldehydes and in other dry foodstuffs in which there are components that are reactive with the dipeptide sweetener, is impeded by problems resulting from the somewhat limited chemical stability of the dipeptide sweeteners. WO-92/07473 and EP-A-0457724, for example, state that aspartame shows instability in the presence of aldehydes, ketones, moisture etc. This is already detrimental during the production of the dry foodstuffs, but also detrimental to the storage stability of the products in which the said components are present together, as the sweetening power decreases as a result and/or part of the desired effect of the other component is lost. Under such circumstances, as a result of unwanted reactions, there may also be discoloration of the (solid) products in which a dipeptide sweetener is incorporated.

For interactions between APM (and Ace-K) and various other substances (such as water-soluble vitamins and food acids, e.g. malic, citric, acetic or tartaric acid) reference is made to: Kroyer G., et al., Ernährung (AUT), 1993, pp. 614-617 (Part II), where stability problems in dry mixtures, in particular, are described, as shown by experiments at increased temperature.

Furthermore, in various dry foodstuffs containing dipeptide sweetener, such as chocolates and hard confectionery, regardless of the presence or absence of components that are reactive with the dipeptide sweetener, distribution of the flavour and sweetness through the foodstuff appears to be non-optimum and more or less inhomogeneous. In the preparation of hard confectionery (such as drops) using, for example, aspartame as a sweetener, handling using state-of-the-art techniques often is difficult and it is necessary to use a previously prepared dispersion of aspartame in the acid components of the drops. If the acid component is absent or present in insignificant amounts, as in hard confectionary with menthol flavour, homogeneous distribution of the dipeptide sweetener is impossible or hardly possible. In that case, the dipeptide sweetener has a high tendency to clumping.

In the prior art efforts have been made to find solutions with the aim of improving the sweetness stability and the flavour of dry foodstuffs sweetened with a dipeptide sweetener with particular regard also to combating stability problems due to the presence of dipeptide sweetener in such foodstuffs, for example in chewing gums, by for example providing the dipeptide sweetener with a coating, in an extra process step. This is described in EP-A-0461197, for example. As described in EP-A-0160607, for example, attempts have also been made to combat stability problems in chewing gums containing aldehydes, for example, by applying the dipeptide sweetener to the surface of the chewing gum. All this has disadvantages, however, in terms of the complexity of manufacture and requires additional process steps. In addition, so-called “hot spots” may occur in dry foodstuffs which are sweetened with coated sweeteners. In order to improve these various aspects, blends of a dipeptide sweetener and another intense sweetener, such as acesulfame-K or saccharin, are therefore also used in dry foodstuffs. When such blends are used, different peaks may occur in the perception of the sweetness during consumption of the dry foodstuff because of differences in the sweetening profiles of the various sweeteners, and there is also a risk that a bitter or other off-flavour may be perceived for certain periods during consumption. In addition it should be noted that attempts have also been made to influence the profile of the release of sweetening power in dry foodstuffs, such as chewing gums etc., by varying the particle size of the dipeptide sweetener used in these products (see for example EP-A-0427541), or by appropriate choice of the location of the sweetener in the dry foodstuff product to be sweetened (e.g. by powdering the outside of the dry foodstuff with sweetener or by providing the dry foodstuff itself with a coating which is also sweetened and optionally contains an aldehyde aroma component, as is described for example in EP-A-0129584).

According to the prior art there is thus still no entirely satisfactory solution for improving the sweetness stability and the flavour of such dry foodstuffs and for limiting the consequences of any discoloration occurring. This is especially the case when there is also reduced storage stability (i.e. loss of sweetening power during storage).

There is therefore a need to provide dry foodstuffs containing dipeptide sweetener with improved sweetness stability, improved flavour quality and reduced susceptibility to discoloration. There is also a need for a method of producing such dry foodstuffs with improved sweetness stability, improved flavour quality and reduced susceptibility to discoloration in a simple way. In particular there is a need to improve the sweetness stability, the flavour quality and resistance to discoloration of dry foodstuffs sweetened with a dipeptide sweetener which also contain flavourings that are reactive with the dipeptide sweetener such as aldehyde-based flavourings.

Surprisingly, extensive research by the applicant has now yielded dry foodstuffs containing dipeptide sweetener with outstanding sweetness stability and flavour quality and with good resistance to discoloration, where the dipeptide sweetener is a sweetening salt of an aspartic-acid-derived dipeptide sweetener and a derivative of a sweetening acid. It has been found that the dry foodstuffs with such a composition show a surprisingly good sweetness stability during storage as well as good flavour quality and are not susceptible to discoloration.

In particular it has also been found that dry foodstuffs sweetened with a dipeptide sweetener which also contain aldehyde-based flavourings that are reactive with the dipeptide sweetener or in which there are present other components that can react with the dipeptide sweetener, show a surprisingly good sweetness stability during storage and are highly resistant to discoloration when the dipeptide sweetener is a sweetening salt of an aspartic-acid-derived dipeptide sweetener and a derivative of a sweetening acid. The storage stability of such products is considerably better than that of dry foodstuffs sweetened with a (free or mixed) dipeptide sweetener.

Sweetening salts of an aspartic-acid-derived dipeptide sweetener and a derivative of a sweetening acid, as meant here, are described in CA-A-1027113 and ES-A-8604766, and also in Belgian patent application No. 9500836, which had not been pre-published (on the priority date of the present application) and which led to inter alia EP-A-0768041. Derivatives of sweetening acids as meant here are (derivates of) organic acids corresponding to an intense sweetener which is not derived from aspartic acid. From none of the aforementioned documents, in which moreover the possibilities of application for the sweetening salts in question are only indicated in very general terms without showing concrete applications, can it be inferred or expected that these sweetening salts, in dry foodstuffs, have such a strong effect on the sweetness stability during storage as well as on the flavour quality and on the resistance to discoloration.

This is relevant not only in comparison with dry foodstuff products in which only a dipeptide sweetener is present as the sweetening component but also in comparison with dry foodstuff products in which the dipeptide sweetener is used in the form of a blend.

Examples of suitable sweetening salts which according to the invention can be used in dry foodstuffs are: salts of aspartame (APM) or alitame (ALI) with acesulphamic acid (i.e. the acid derived from Ace-K), with saccharic acid (1,2-benz-isothiazol-3(2H)-one-1,1 dioxide) and with cyclohexylsulphamic acid (cyclamate). The most suitable one is the salt of aspartame and acesulphamic acid, hereinafter also referred to as APM-Ace or as the APM-Ace salt; particularly suitable is the exceptionally stable, non-hygroscopic product as obtained in solid form by the process in Belgian non-prepublished patent application No. 9500836. In that process, aspartame and a salt of acesulphamic acid are allowed to react in an aqueous medium in the presence of a strong acid and the APM-Ace formed is isolated from the reaction mixture.

The particle size of the sweetening salts which under this invention are used in the relevant dry foodstuffs, and in particular the particle size of the sweetening salts as present at the time of manufacture of the dry foodstuffs, is not particularly critical, but is generally not more than 1000 μm. The presence of larger particles in the dry foodstuff causes less homogeneous and less balanced release of the sweetening power during consumption. Use of a sweetening salt with a particle size <350 μm offers advantages in terms of the flavour quality of the dry foodstuffs. The best flavour quality is achieved if the particle size of the sweetening salt is in the range <100 μm, but particles in the range from 100-200 μm also give products with excellent sweetness stability and storage stability when used in dry foodstuffs. In a number of cases somewhat better stability of the products is obtained with sweetening salt particles in the range from 100-200 μm. By choosing the particle size of the sweetening salt to be used, the specialist can achieve further fine-tuning of the desired flavour quality and stability.

In the manufacture of dry foodstuffs according to the invention, use is preferably made of sweetening salts in substantially dry form. “In substantially dry form” means that the salt, as available in dry form (i.e. with a moisture content of 2% by wt or less), is directly incorporated into the dry foodstuff, for example by mixing the dry ingredients, with or without subsequent compacting, etc. It is also possible to use the sweetening salt in substantially dry form by moistening it to a limited extent, for example in a wet granulation step, or even incorporating it in the form of a wet slurry, in which at least 85% by wt of the original sweetening salt is still present in solid form, and processing the product so obtained into the final product in the usual way, by (spray) drying, agglomeration and, if necessary, grinding, etc.

The content of sweetening salt in the dry foodstuffs according to the invention can vary within a very wide range; for chewing gums it should generally lie within the range from 100 to 6000 ppm (calculated relative to the total mass of the chewing gum); for table sweeteners the content of sweetening salt in the sweetener may be in the range from 10 to 50% by wt (calculated relative to the total sweetener). Other ranges may be applicable for other products. These can easily be determined by the specialist, depending on the desired sweetness etc. of the products. In chocolates and other dry confectionery, for example, the sweetening salt content will usually be from 400 to 3000 ppm. The invention is in addition particularly advantageous in the manufacture of hard confectionery containing no or only little nutritive acid and where the transparency of the confectionery also plays a role. It has been found that when the process of the invention is used for the manufacture of transparent, hard confectionery, such as mint sweets or medicinal mentholyptus sweets, completely clear and transparent sweets can be obtained as a result of the excellent dispersability of the sweetening salts used, the same proving impossible when blends of the individual components of the said salts are used.

Also, the taste and oral sensation of the sweets obtained according to the invention are clearly better and more similar to those of sweets sweetened with sugar than of sweets obtained using blends of the individual components. In the manufacture of sugar-free chocolates, too, the present invention has proved particularly suitable; the chocolates obtained with it have a sweetness resembling that of sugar more closely and a fuller taste than chocolates obtained in like fashion from blends of the individual components.

It will be clear to the specialist that although dry foodstuffs with excellent sweetness stability and storage stability are already produced according to the invention, further adjustments with regard to the flavour profile and other properties of the foodstuffs, such as the said stabilities, can be made by adding additional small quantities of a separate intense sweetener or of a blend of sweeteners, in addition to choosing the particle size of the sweetening salt. Accelerated emergence of the sweet flavour, the so-called “up-front release”, can thus be achieved, for example. In addition, the properties in question can be further influenced by applying all or a proportion of the particles of the sweetening salt in the dry foodstuff in coated form, or by adding the additional small quantity of a separate intense sweetener (if one is used) to the dry foodstuff also partially or entirely in coated form. Application of part or all of the sweetener in coated form ensures an adapted profile of the release of the sweet flavour and can thus be regarded as a form of “controlled release”. In addition, all the known techniques in relation to the technical shaping of the final products (the dry foodstuffs) and/or the components to be incorporated, such as the sweetening salt, are of course also available to the specialist in order to achieve further desired flavour effects and/or aesthetic effects. Examples of such techniques in relation to the technical shaping of the components are granulation, grinding, freeze-drying, spray-drying, agglomeration etc. The invention is in no way limited with regard to the place where the sweetening salt used according to the invention is located in or on the dry foodstuff.

Where the dry foodstuff relates to chewing gums, it should be noted that chewing gums, as is known and customary, consist in large part (about 5 to 95% by wt) of a so-called “gum base” and all sorts of additives, such as aroma substances. The gum base generally consists of a combination of natural gums and/or synthetic elastomers and resins, and also contains softeners and (5 to 60% by wt) inorganic fillers. The gum base may also contain other components such as antioxidants, colourants and emulsifiers. Furthermore, chewing gums contain all sorts of flavourings which may originate from natural plant extracts or be obtained synthetically.

Examples of aldehyde flavourings which can be used in dry foodstuffs are acetaldehyde, benzaldehyde, anisaldehyde, cinnamaldehyde, citral, α-citral, β-citral, decanal, ethyl vanillin, heliotropin (piperonal), vanillin, α-amyl-cinnamaldehyde, citronellal, aldehyde C-8, aldehyde C-9, aldehyde C-12, trans-2-hexenal, veratraldehyde, etc.

Examples of ketones which can be used in dry foodstuffs are acetophenone, acetone, methyl-n-amylketone, p-hydroxyphenyl-2-butanone, α-ionone, β-ionone, methyl-3-butanone, 2-heptanone, o-hydroxy-acetophenone, 2-methyl-2-hepten-6-one, 2-octanone, 2-undecanone and 2-pentanone.

Besides the sweetening salts to be used according to the invention, food sugars and/or other sweeteners can also be present in the dry foodstuffs according to the invention. Examples of these are aspartame, alitame, acesulfame-K, saccharinates, cyclamates, glycyrrhizin, thaumatin, monellin, neohesperidin dihydrochalcone (NHDC), sucralose, sorbitol, mannitol, xylitol, lactitol, isomalt, maltitol, hydrogenated starch hydrolysates, dextrins, sucrose, dextrose, glucose syrups, fructose, fructose syrups, etc., or combinations thereof.

The invention will now be clarified by means of some tests and comparative tests, in which chewing gums, chocolates and hard confectionery are used as an example of dry foodstuffs, but is in no respect limited to these.

The sweetening salt of aspartame and acesulphamic acid (hereinafter referred to as APM-Ace) used in the investigations was produced by the method in Experiment 4a of Belgian non-prepublished patent application No. 9500836. The procedure was as follows:

608 g of APM and 410 g of AceK (each 2.0 mol) were added consecutively to 2 l of water at 20° C., after which the resultant slurry was brought to 50° C. and 370 g of 20% HCl in water was added, with stirring, over 30 minutes. In this operation the initially rather voluminous slurry changed into a less voluminous slurry. At the end of the addition of HCl the slurry obtained was cooled to 10° C. over about 30 minutes via indirect heat exchange with a cooling medium at 0°C. and was then filtered off. The crystalline mass obtained was washed with a minimum quantity of chilled water and dried in a vacuum at 40° C. This produced in total 820 g (i.e. a yield of 90%) of a white product which according to ¹H-NMR consisted entirely of the 1:1 salt of APM and acesulphamic acid, with a purity >99%. The moisture content was 0.12%. Two fractions were separated from this product by sieving procedures: an initial fraction with particles <100 μm and a fraction with particles of 100-200 μm.

For comparison purposes corresponding particle size fractions were also made from each of the sweeteners aspartame and acesulfame-K separately, and 1:1 blends were made up on a molar basis both for APM and Ace-K with a particle size <100 μm and for APM and Ace-K with a particle size in the range 100-200 μm. These blends had a composition of approximately 3:2 by weight. When they were used in the comparative experiments, the quantity of the blends taken in each case was such that (allowing for corrections for the potassium content in Ace-K and for the water content, determined by the “loss-on-drying” method, in APM) the same amount of aspartame was used as in the case of addition of the sweetening APM-Ace salt.

As part of the investigation, tests (and comparative tests) were made with chewing gums with cinnamon flavour in order to determine various sensory properties (“attributes”), and in addition analytical tests were made with regard to the storage stability of the chewing gums. All the chewing gums tested were made in accordance with a standard formulation consisting of a sugar-free gum base and sorbitol. The formulations conformed to the following composition:

| | Ingredients |
|---|---|
| | (% w/w): |
| sorbitol powder (P100T, Roquette) | 52.4 |
| sugar-free gum base (Valencia-T, Cafosa) | 24.6 |
| sorbitol solution (Neosorb 70/70; Roquette) | 16.2 |
| glycerine | 4.8 |
| cinnamon flavour (17.42.7305, IFF) | 2.0 |
| | 100.0 |

2700 ppm of the sweetener APM-Ace to be used according to the invention was also incorporated in this composition, in two separately tested fractions in terms of particle size, viz. a fraction <100 μm (see tests K1) and a fraction of 100-200 μm (see tests K2).

For comparison purposes, tests were made with compositions as indicated above in which the 2700 ppm of APM-Ace was replaced by 3000 ppm of a blend of APM and Ace-K (in a 1:1 molar ratio; 3:2 ratio by weight). In determining the amount of 3000 ppm allowance was made for the correction for the water content of APM (according to the “loss-on-drying” method) and the potassium content of Ace-K. In order to optimize comparability, use was also made of blends in two different fractions in terms of particle size: a fraction with APM and Ace-K particles <100 μm (see comparative tests K1A) and a fraction with APM and Ace-K particles of 100-200 μm (see comparative tests K2A).

The procedure for making the test chewing gums (including that for the comparative test chewing gums KA) was in each case as follows:

The gum base was put into a Z-blade mixer and softened in it with heating to a final temperature of 50 to 60° C. and stirring. In each formulation the sweetener in the amount indicated (see above) was first dispersed in the sorbitol powder, while the sorbitol solution with the glycerine was also heated, to 60° C., before the sorbitol powder and sorbitol solution were both added to the gum base, with stirring. The whole was mixed for 10 minutes at the above-mentioned temperature, and then for a further 10 minutes with the heating element switched off. The dough obtained was rolled out with a dough roller (“pastry brake”) to a thickness of 1.5 mm. Strips of 73×19 mm, which were each packed in foil (as is usual for chewing gum), were cut out of the rolled-out mass.

Part of the chewing gums made in this way was then kept frozen in order that samples which best approximated to the original condition of the relevant chewing gum would be available for comparison at any desired time. Another part of the chewing gums with cinnamon flavour was stored at 20° C. (storage-quality tests). Sensory tests were made on these chewing gums after 4 and 12 weeks of storage, with comparisons being made with corresponding chewing gums that had been kept frozen at −18° C. Yet another part of the chewing gums with cinnamon flavour was kept at 40° C., as an accelerated storage-quality test. The storage stability of these chewing gums was monitored by analysis of the content of APM and Ace-K (or Ace) after 14 and 28 days of storage.

As regards the sensory tests, the chewing gums were evaluated by an experienced panel, with 6 members, for:

sweetness intensity, hereinafter referred to as “SI”;

sweetness quality, hereinafter referred to as “SQ”, particular attention being paid to off-flavours such as metallic flavour and bitterness;

release of the desired flavour (here mint and cinnamon flavour, respectively), hereinafter referred to as “FR”; and for

overall acceptability of the chewing gum, hereinafter referred to as “OA”,

these evaluations in each case being made after 30 seconds, 2 minutes, 6 minutes, 10 minutes and 15 minutes of chewing.

In the sensory tests with the cinnamon flavour chewing gums, the products according to the invention scored better in all cases than the corresponding comparison products. Both in the comparison products and in the products according to the invention, however, a deterioration of attributes as a result of storage at 20° C. was observed. In all the products there was slight discoloration relative to the products which had been kept under deep-freeze conditions. When the cinnamon chewing gum products sweetened with the sweeteners <100 μm and 100-200 μm were compared with each other, the former products were found to deteriorate somewhat more in terms of sweetness than the latter, but in both types of product there was perceptible sweetness and flavour throughout the 15 minutes of the sensory tests. The overall flavour score for the products which were sweetened with the smaller particles was somewhat higher than for the products which were sweetened with the larger particles. In the comparison chewing gums with cinnamon flavour (in the samples tested after 4 weeks) there was no longer any perceptible sweetness or flavour after about 6-8 minutes.

The sensory findings for the cinnamon flavour products, as evaluated after 4 and 12 weeks of storage, are summarized in Tables I and II below. The bottom row in these tables shows the differences from the frozen chewing gums.

TABLE I
Storage time 4 weeks at 20° C.

| | TEST K1 | COMP. TEST | TEST K2 | COMP. TEST |
|---|---|---|---|---|
| SI | faster start | slow but | slow start; | faster |
| | than K1A; | good in | slow build- | build-up of |
| | good build-up | terms of | up of | sweetness |
| | of sweetness; | build-up; | sweetness; | than K1A; |
| | peak after 2 | peak after | 2peak after 2 | somewhat |
| | minutes; | minutes; | minutes; | hotter |
| | then decrease | then marked | decrease in | taste than |
| | in sweetness; | decrease in | sweetness | K1 or K1A; |
| | sweetness | sweetness; | but less | fast |
| | remains | still | than K2A | decrease; |
| | clearly | slightly | (and than K1 | scarcely |
| | perceptible | sweet after | and K1A); | any |
| | for more than | 6 minutes; | lengthened | sweetness |
| | 15 minutes | flat flavour | sweetness | left at 4 |
| | | after 10 | and flavour | minutes; |
| | | minutes | profile for | scarcely |
| | | | more than 15 | any flavour |
| | | | minutes | left after |
| | | | | 8 minutes |
| SQ | good quality | good, but | good; no | good, but |
| | and good | perceptible | off-taste | perceptible |
| | persistence; | for too | perceptible; | for too |
| | no off-taste, | short a | not bitter | short a |
| | not bitter; | time; no | | time; no |
| | better than | off-taste, | | off-taste; |
| | K1A | not bitter | | not bitter |
| FR | good; sweet- | good, but as | good; reten- | good, but |
| | ness and fla- | short as | tion of fla- | somewhat |
| | your remain | sweetness | your because | sharper than |
| | clearly per- | | of | K1A; as |
| | ceptible | | continuing | short as |
| | | | sweetness | sweetness |
| OA | markedly | reasonable | markedly | reasonable |
| | better than | | better than | |
| | K1A; | | K2A | |
| | somewhat | | | |
| | better than | | | |
| | K2 | | | |
| *) | as sweet | less intens- | slower start | slower start |
| | after 2 min- | ely sweet | to sweetness | to sweetness |
| | utes; some- | and less | build-up; | build-up |
| | what faster | flavour than | somewhat | |
| | decrease in | REF | later but | |
| | sweetness | | equally | |
| | relative to | | strong peak | |
| | REF | | | |

*)relative to product stored at −18° C. (REF)

TABLE II
Storage time 12 weeks at 20° C.

| | | COMP. TEST | | COMP. TEST |
|---|---|---|---|---|
| | TEST K1 | K1A | TEST K2 | K2A |
| SI | rather slow | slow but | slower start; | slow start; |
| | start; | build-up | than K2A; | build-up of |
| | good build-up | after 30 | slow build- | sweetness |
| | from 40 sec | sec; peak | up of sweet- | to peak |
| | onwards; peak | after 2 | ness; peak | after 2 |
| | after 2 | minutes; | after 2.5 | minutes; |
| | minutes; | then marked | minutes; | rapid |
| | rather less | decrease in | decrease in | decrease; |
| | than for K1A; | sweetness; | sweetness, | scarcely |
| | then decrease | still | but less | any sweet- |
| | in sweetness | slightly | than K2A; | ness left |
| | but not as | sweet after | lengthened | at 3.5 min- |
| | fast as K1A; | 4 minutes; | flavour and | utes; scar- |
| | sweetness and | very flat | sweetness | cely any |
| | flavour | flavour | profile for | flavour |
| | remain for | after 6 | more than 15 | left after |
| | more than 15 | minutes; an | minutes | 6 minutes; |
| | minutes | effort to | | no sweet- |
| | | continue | | ness or |
| | | chewing | | flavour |
| | | | | perceptible |
| | | | | after 10 |
| | | | | minutes |
| SQ | reasonable | reasonable, | reasonable; | reasonable |
| | quality and | but very | not bitter | beginning, |
| | good | sharp | but percep- | |
| | persistence; | refreshing | tible for | |
| | not bitter; | taste at | too short a | |
| | better than | the back of | time; not | |
| | K1A | the tongue; | bitter | |
| | not bitter | | | |
| FR | flavour | peak moderate | reasonable; | reasonable, |
| | after 2 min- | with dull | retention of | but sharp |
| | utes; | flavour, but | flavour | burnt fla- |
| | reasonable; | as short as | because of | your at |
| | sweetness and | sweetness | continuing | beginning; |
| | flavour | | sweetness- | flavour and |
| | remain clear- | | somewhat | sweetness |
| | ly percep- | | flatter than | present for |
| | tible | | K2A | too short a |
| | | | | time |
| QA | markedly | reasonable | remains | reasonable |
| | better than | | sweet and | |
| | K1A | | tasty for 15 | |
| | | | min | |
| *) | rather more | rather more | rather more | rather more |
| | discoloured; | discoloured; | discoloured; | discol- |
| | sweetness | more refres- | slower | oured; swe- |
| | profile iden- | hing; | build-up of | etness |
| | tical but | sweetness | sweetness | intensity |
| | rather lower | profile ide- | and rather | much less, |
| | intensity and | ntical but | less | faster |
| | after 10 min- | intensity | intensely | decrease in |
| | utes rather | much lower; | sweet; | sweetness |
| | less flavour | flavour less | rather less | and flavour |
| | | fresh | flavour, but | |
| | | | better than | |
| | | | K2A | |

*)relative to product stored at −18° C. (REF)

The results of the sensory tests made with the cinnamon (aldehyde) flavour test chewing gums and comparison chewing gums were also confirmed by accelerated aging tests (at 40° C.), in which the contents of APM and Ace-K (or Ace) were determined by HPLC (high-pressure liquid chromatography). An overview of the relevant HPLC analysis results is given in Table III below:

TABLE III
Relative decrease in contents of APM and
Ace-K or Ace during storage
at 40° C. for:

| | | | 14 days | 28 days |
|---|---|---|---|---|
| | | 0 days | residual | residual |
| | | content | content | content |
| sample | comp. | (%) | (%) | (%) |
| <100 μm | | | | |
| K1 | APM | 100 | 47 | 36 |
| | Ace | 100 | 100 | 95 |
| K1A | APM | 100 | 10 | 0 |
| | Ace-K | 100 | 94 | 95 |
| 100-200 μm | | | | |
| K2 | APM | 100 | 45 | 44 |
| | Ace | 100 | 83 | 84 |
| K2A | APM | 100 | 16 | 0 |
| | Ace-K | 100 | 96 | 100 |

In the framework of the examination, tests (and comparative tests) were also conducted on sugar-free chocolates and hard confectionery for the purpose of establishing various sensory properties (attributes).

The preparation of sugar-free chocolates and comparative chocolates started from unsweetened chocolates made on the basis of palatinite without addition of an intense sweetener. The unsweetened chocolate in question was included in the tests as a basis for comparison. Also, from it were prepared basis for comparison. Also, from it were prepared batches of a chocolate sweetened in accordance with the invention (chocolate I) and of a comparative chocolate sweetened with a blend of sweeteners (chocolate A). This was done by carefully melting the chocolate and mixing the sweetener, in the desired quantity, through the molten chocolate. 800 ppm of APM-Ace (0-100 μm fraction) was added to chocolate I; a blend of 537 ppm of APM and 351 ppm of Ace-K (0-100 μm fraction of each) were added to comparative chocolate A. The amounts in question, given their theoretical equivalence in sweetening power, are deemed to be equally sweet. No differences in handleability were observed in the preparation. The chocolate and the comparative chocolate were judged by an experienced panel of tasters (n=4) in terms of the intensity and quality of the sweetening power and flavour as well as in terms of any aftertaste and acceptability. Chocolate I was judged to be significantly better than comparative chocolate A. Chocolate I had a full, round taste (which moreover soon emerged) with good sweetness and associated flavour properties and excellent acceptability; the chocolate did not produce any greasy gustatory effect. In the case of the comparative chocolate, in contrast, the sweetness and taste emerged more slowly and were not so full and round than of chocolate I; moreover, the gustatory effect was rather greasy and acceptability was poor.

In the preparation of sugar-free, hard confectionery (and associated comparative products), products with a minth taste and with a medicinal mentholyptus taste were prepared.

This preparation started from palatinite and maltitol as a starting composition. This starting composition was sweetened batch by batch for the preparation of sugar-free, hard confectionery, more specifically a confectionery sweetened in accordance with the invention (confectionery I) and of a comparative confectionery sweetened with a blend of sweeteners (confectionery A). This was done by adding the sweetener directly to the hot starting composition of the mass of confectionery, while stirring, in order to disperse the sweetener. For confectionery I, 1000 ppm of APM-Ace (100-200 μm fraction) was added; for comparative confectionery A, a blend of 672 ppm of APM and 438 ppm of Ace-K (100-200 μm fraction of each) was added. The amounts in question, given their theoretical equivalence in sweetening power, are deemed to be equally sweet. A significant difference was observed, however, between confectionery I and confectionery A. In the preparation, the sweetener in confectionery I dispersed more rapidly than the sweetener in confectionery A. Also, the sweetener in comparative confectionery A formed clumps whilst the sweetener in confectionery I dissolved to leave a completely clear solution. Confectionery I and comparative confectionery A were judged by an experienced panel of tasters (n=4) in terms of the intensity and quality of the sweetening power and flavour as well as in terms of any aftertaste and acceptability. Confectionery I was judged to be significantly better than comparative confectionery I. Hard confectionery I had excellent acceptability with good sweetness intensity and related flavour properties; the flavour, too, had good intensity. In the case of comparative confectionery A, in contrast, the sweetness emerged more slowly and was less intense than in the case of confectionery I; moreover, the flavour was unbalanced and less acceptable. The two batches of sweets obtained also showed significant differences in appearance. The sweets from confectionery I were completely clear and transparent, those from confectionery A were not.

The presence of sweetening salts in dry foodstuffs can be determined by IR-spectroscopic techniques.

1. Dry foodstuffs containing dipeptide sweetener, characterized in that the dipeptide sweetener is a sweetening salt of an aspartic acid based dipeptide sweetener and a derivative of a sweetening acid.

2. Dry foodstuffs containing dipeptide sweetener and a component that is reactive with it, characterized in that the dipeptide sweetener is a sweetening salt of a dipeptide sweetener derived from aspartic acid and a derivative of a sweetening acid.

3. Dry foodstuffs according to claim 2, characterized in that the dry foodstuff containing dipeptide sweetener also contains an aldehyde flavouring substance.

4. Dry foodstuff according to claims 1 through 3, characterized in that the sweetening salt is chosen from the salts of aspartame with acesulphamic acid, saccharinic acid or cyclohexylsulphamic acid and from the salts of alitame and acesulphamic acid, saccharinic acid or cyclohexylsulphamic acid.

5. Dry foodstuff according to claim 4, characterized in that the sweetening salt is the salt of aspartame and acesulphamic acid.

6. Dry foodstuff according to claim 5, with the salt of aspartame and acesulphamic acid having been obtained by allowing aspartame and a salt of acesulphamic acid to react in an aqueous medium in the presence of a strong acid and isolating the salt formed from the reaction mixture.

7. Dry foodstuff according to any one of claims 1-6, characterized in that the particle size of the sweetening salt is smaller than 1000 μm, in particular smaller than 350 μm, more preferably smaller than 200 μm and most preferably is in the range below 100 μm.

8. Dry foodstuff according to any one of claims 1-7, characterized in that the taste profile of the dry foodstuff has been adapted yet further due to at least part of the sweetening salt having been applied in coated form or in an appropriate technical shape or due to further amounts of a sweetener having been added to the dry foodstuff at one or more suitable places in it or due to the technical shape of the dry foodstuff having been suitably adapted.

9. Process for the preparation of dry foodstuffs containing dipeptide sweetener, characterized in that the dipeptide sweetener used in the preparation of the dry foodstuff is a sweetening salt in substantially solid form of an aspartic acid based dipeptide sweetener and a derivative of a sweetening acid.

10. Process for the preparation of dry foodstuffs containing dipeptide sweetener and a component that is reactive with it, characterized in that the dipeptide sweetener used in the preparation of the dry foodstuff is a sweetening salt in substantially solid form of an aspartic acid based dipeptide sweetener and a derivative of a sweetening acid.

11. Process according to claim 10, characterized in that the dry foodstuff containing dipeptide sweetener also contains an aldehyde flavouring substance.

12. Process according to any one of claims 9 through 11, characterized in that the sweetening salt used is chosen from the salts of aspartame with acesulphamic acid, saccharinic acid or cyclohexylsulphamic acid and from the salts of alitame with acesulphamic acid, saccharinic acid or cyclohexylsulphamic acid.

13. Process according to claim 12, characterized in that the sweetening salt used is the salt of aspartame and acesulphamic acid.

14. Process according to claim 13, wherein a salt of aspartame and acesulphamic acid in solid form is used which has been obtained by allowing aspartame and a salt of acesulphamic acid to react in an aqueous medium in the presence of a strong acid and isolating the salt formed from the reaction mixture.

15. Process according to any one of claims 9-14, characterized in that the particle size of the sweetening salt is smaller than 1000 μm, in particular smaller than 350 μm, more preferably smaller than 200 μm and most preferably is in the range below 100 μm.

16. Process according to any one of claims 9-15, characterized in that at least part of the sweetening salt used has been applied in coated form or in an appropriate technical shape or with further amounts of a sweetener being added to the dry foodstuff or with the technical shape of the dry foodstuff being suitably adapted.

17. Dry foodstuffs and process for the preparation thereof as substantially described in the specification and the examples.
